(19) United States
(12) Patent Application Publication
(10) Pub. No.: US 2025/0090066 A1
(43) Pub. Date: Mar. 20, 2025

(54) Automated Urinary Output Monitoring System

(71) Applicant: C. R. Bard, Inc., Franklin Lakes, NJ (US)

(72) Inventor: Michal Tourchak, Atlanta, GA (US)

(21) Appl. No.: 18/701,916

(22) PCT Filed: Oct. 17, 2022
(86) PCT No.: PCT/US22/46920

§ 371 Date: Apr. 16, 2024

Related U.S. Application Data
(60) Provisional application No. 63/271,591, filed on Oct. 25, 2021.

(51) Int. Cl. A61B 5/20

(52) U.S. Cl. CPC A61B 5/208; A61B 2560/0443; A61B 2562/0252

(57) ABSTRACT

An automated urinary output monitoring system includes a urine collection bag in fluid communication with a urinary catheter and an automated urinary output monitoring device. The automated urinary output monitoring device is coupled to a securing surface and includes a load cell assembly in communication with a console. The load cell assembly is configured to detect one or more load values when urine collection bag is coupled to the automated urinary output monitoring device. The automated urinary output monitoring device further includes a coupling system configured to detachably couple the urine collection bag to the automated urinary output monitoring device. The coupling system includes a slide track configured to slidably receive a spine, and a locking mechanism configured to transition between an unlocked configuration and a locked configuration.

### PRIORITY

This application claims the benefit of priority to U.S. Provisional Application No. 63/271,591, filed Oct. 25, 2021, which is incorporated by reference in its entirety into this application.

### BACKGROUND

Urine output monitoring systems may be used to monitor the urine output of a patient. Some current systems use a weight based approach to monitor the volume of urine and the rate of collection. However, the coupling system that detachably couples a urine collection bag to a monitoring device can be bulky, impeding the ability of clinicians to perform necessary tasks. Furthermore, patient data including patient identification may be stored on the coupling system requiring the coupling system and the urine collection bag to travel with the patient. It would be beneficial to the clinician and the patient to have an automated urine output monitoring system that has a compact coupling system that stores patient information, allowing the urine collection bag and coupling system to more efficiently travel with the patient. Disclosed herein are an automated urinary output monitoring system and method of use that address the foregoing.

### SUMMARY

Disclosed herein is an automated urinary output monitoring system that, according to some embodiments, includes (i) a urine collection bag configured to collect urine excreted by a patient via a urinary catheter and (ii) an automated urinary output monitoring device. The automated urinary output monitoring device includes a load cell assembly operatively coupled with the urine collection bag, where the load cell assembly is configured to measure a load defined by urine collected within the urine collection bag. The automated urinary output monitoring device includes a console coupled with the load cell assembly, where the console includes a processor and a non-transitory computer readable medium having logic stored thereon that, when executed by the processor, performs operations that include determining a volume of the urine collected with the urine collection bag based on the load. The automated urinary output monitoring system further includes a coupling system configured to detachably couple the urine collection bag to the automated urinary output monitoring device, where the coupling system includes a (i) rail attached to one of the urine collection bag or the automated urinary output monitoring device and (ii) a channel attached to the other one of the urine collection bag or the automated urinary output monitoring device. The channel is configured to (i) longitudinally and slidably receive the rail and (ii) prevent lateral separation of the rail from the channel.

In some embodiments, the channel is attached to a front side of the automated urinary output monitoring device and the rail is attached to a back side the urine collection bag.

In some embodiments, the rail extends laterally across a portion of a width of the urine collection bag and in some embodiments, the channel extends laterally across the front side of the automated urinary output monitoring device.

In some embodiments, the channel includes an open end and a closed end and in some embodiments, the channel includes a bumper at the closed end, where the bumper is configured to abut the rail when the rail is received within the channel.

In some embodiments, the coupling system includes a locking mechanism configured to transition between an unlocked configuration and a locked configuration such that (i) longitudinal displacement of the rail with respect to the channel is restricted when the locking mechanism is transitioned to the locked configuration, and (ii) longitudinal displacement of the rail with respect to the channel is allowed when the locking mechanism is transitioned to the unlocked configuration.

In some embodiments, the locking mechanism includes a deflectable protrusion coupled with the channel and a recess disposed within the rail, where the recess is configured to receive the protrusion when the locking mechanism is transitioned to the locked configuration, thereby restricting longitudinal displacement of the rail with respect to the channel.

In some embodiments, the locking mechanism includes an electro-mechanical actuator operatively coupled between the deflectable protrusion and the console, and the operations further include activating the electro-mechanical actuator to (i) transition the locking mechanism from the unlocked configuration to the locked configuration and (ii) transition the locking mechanism from the locked configuration to the unlocked configuration. In some embodiments, the locking mechanism includes a latch member rotatably coupled to the channel at the open end. In such embodiments, the latch member is rotated so as to extend across the open end when the locking mechanism is transitioned to the locked configuration, and the latch member is rotated so as to extend away from the open end when the locking mechanism is transitioned to the unlocked configuration. In some embodiments, locking mechanism includes an electro-mechanical actuator operatively coupled between the latch member and the console, the operations further include activating the electro-mechanical actuator to (i) transition the locking mechanism from the unlocked configuration to the locked configuration and (ii) transition the locking mechanism from the locked configuration to the unlocked configuration.

In some embodiments, the rail includes a patient identification device coupled thereto, the patient identification device having patient stored in memory thereon.

In some embodiments, the patient identification device includes a printed circuit board including a number of electrical contacts disposed along the rail, and the channel includes a console connector coupled with the console, where the console connector includes a number of connector contacts disposed along the channel. In such embodiments, the electrical contacts and the connector contacts are configured to correspondingly couple with each other when the urine collection bag is coupled with the automated urinary output monitoring device to enable data exchange between the console and patient identification device.

In some embodiments, the system further includes a display coupled with the console, and the operations further include depicting on the display the volume of the urine collected with the urine collection bag.

Also disclosed herein is a method of monitoring urinary output that, according to some embodiments, includes (i) coupling a urine collection bag to an automated urinary output monitoring device; (ii) transitioning a locking mechanism from an unlocked configuration to a locked configuration, where the locking mechanism is configured to prevent decoupling of the urine collection bag from the automated urinary output monitoring device in the locked configuration; (iii) determining a volume of urine collected within the urine collection bag; (iv) transitioning the locking mechanism from the locked configuration to the unlocked configuration; and (v) decoupling the urine collection bag from automated urinary output monitoring device.

In some embodiments of the method, coupling the urine collection bag to the automated urinary output monitoring device includes slidably advancing a rail attached to the urine collection bag longitudinally within a channel attached to the automated urinary output monitoring device, where the channel is configured to prevent lateral separation of the rail from the channel.

In some embodiments of the method, the channel is attached to a front side of the automated urinary output monitoring device and the rail is attached to a back side the urine collection bag.

In some embodiments of the method, the locking mechanism is configured to (i) restrict longitudinal displacement of the rail within the channel when the locking mechanism is transitioned to the locked configuration and (ii) allow longitudinal displacement of the rail within channel when the locking mechanism is transitioned to the unlocked configuration.

In some embodiments of the method, the locking mechanism includes a deflectable protrusion coupled with the channel and a recess disposed within the rail, where the recess is configured to receive the protrusion when the locking mechanism is transitioned to the locked configuration, thereby restricting longitudinal displacement of the rail within the channel.

In some embodiments of the method, the locking mechanism includes a latch member rotatably coupled to the channel at an open end of the channel opposite a closed end of the channel. In such embodiments, (i) the latch member is rotated so as to obstruct the open end when the locking mechanism is transitioned to the locked configuration, thereby securing the rail within the channel, and (ii) the latch member is rotated so as to extend away from the open end when the locking mechanism is transitioned to the unlocked configuration, thereby allowing extraction of the rail from the channel via the open end.

In some embodiments of the method, the urine collection bag includes a patient identification device having a printed circuit board that includes a number of electrical contacts disposed along the rail and the automated urinary output monitoring device includes a console coupled with a console connector having a number of connector contacts disposed along the channel. In such embodiments, the electrical contacts and the connector contacts are configured to correspondingly couple with each other when the urine collection bag is coupled with the automated urinary output monitoring device to enable data exchange between the console and patient identification device, and the method further includes obtaining patient data from the patient identification device.

In some embodiments of the method, the automated urinary output monitoring device includes a display coupled with the console, and the method further includes depicting on the display the volume of the urine collected with the urine collection bag.

These and other features of the concepts provided herein will become more apparent to those of skill in the art in view of the accompanying drawings and following description, which describe particular embodiments of such concepts in greater detail.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates a cross-sectional side view of an automated urinary output monitoring system, in accordance with some embodiments;

FIG. 2A illustrates a plan view of a back side of a urine collection bag including a first portion of a coupling system of the system of FIG. 1, in accordance with some embodiments;

FIG. 2B illustrates a plan view of a front side of an automated urinary output monitoring device including a corresponding second portion of the coupling of the system of FIG. 1, in accordance with some embodiments;

FIGS. 3A-3D are plan views of the system of FIG. 1 illustrating an exemplary method of detachably coupling the urine collection bag to the automated urinary output monitoring device, in accordance with some embodiments;

FIGS. 4A-4D illustrate a cross-sectional top views of the locking mechanism of FIGS. 2A-2B transitioning between the unlocked configuration and the locked configuration, in accordance with some embodiments;

FIGS. 5A-5C illustrate a cross-sectional side views of the coupling system including another embodiment of a locking mechanism transitioning from the locked configuration to the unlocked configuration, in accordance with some embodiments; and

FIG. 6 illustrates a flow chart of an exemplary method of monitoring urinary output, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

Unless defined otherwise, all technical and scientific terms used herein have the same meaning as commonly understood by those of ordinary skill in the art.

The phrases “connected to,” “coupled to,” and “in communication with” refer to any form of interaction between two or more entities, including but not limited to mechanical, electrical, magnetic, electromagnetic, fluid, and thermal interaction. Two components may be coupled to each other even though they are not in direct contact with each other. For example, two components may be coupled to each other through an intermediate component.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC”, etc.), a semiconductor memory, or combinatorial elements.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

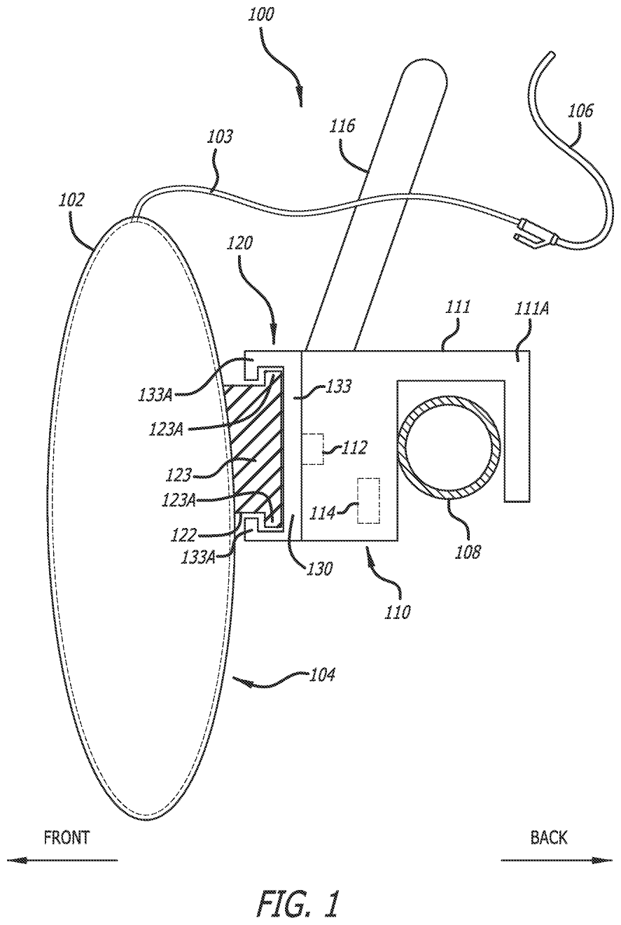

FIG. 1 illustrates a cross-sectional side view of an automated urinary output monitoring system 100, in accordance with some embodiments. In some embodiments, the automated urinary output monitoring system (“system”) 100 includes a urine collection bag (“bag”) 102 having a drainage tube 103 configured to fluidly couple the bag 102 with a urinary catheter 106. In some embodiments, the system 100 may include the urinary catheter 106. The bag 102 is detachably coupled with an automated urinary output monitoring device (“device”) 110 which defines a frame 111. In some embodiments, the frame 111 may include an attachment member 111A (e.g., a hook or a clamp) configured to secure the device 110 to a support structure 108 (e.g., a hospital bed, an IV stand, or the like). The bag 102 is configured to receive and collect urine excreted from a patient via the urinary catheter 106. The device 110 includes a load cell assembly 112 configured to measure a load defined by weight of urine collected within the bag 102. The load cell assembly 112 is operatively coupled with a console 114 configured to receive a load measurement from the load cell assembly 112. In some embodiments, the device may include (or be coupled with) a display 116.

The console 114 may include a microprocessor and logic stored in a non-transitory computer-readable storage medium (memory). The console 114 is configured to receive the load measurement from the load cell assembly 112 and the logic is configured to calculate a volume of the urine collected in the bag 102 based on the load measurement. In some embodiments, the logic may also calculate a flow rate of the urine based on a multiple load measurements. The console 114 is communicatively coupled with the display 116 and the logic is configured to depict the volume of urine collected in the bag 102 on the display 116. In some embodiments, the display 116 may be detachably coupled device 110. In some embodiments, the display 116 may include external computing device, such as a tablet or an electric medical record system.

The bag 102 is attached to the device 110 via a coupling system 120. In some embodiments, the coupling system 120 may be configured to suspend the bag from the device 110. The coupling system 120 is configured to enable detachment of the bag 102 from the device 110. The coupling system 120 is configured to operably couple the bag 102 to the load cell assembly 112 so that the load cell assembly 112 may accurately measure the weight of the bag 102 including the collected urine.

The coupling system 120 includes a bag coupling member 122 disposed on a back side 104 of the bag 102 and a corresponding device coupling member 130 disposed on front side of the device 110. In the illustrated embodiment, the bag coupling member 122 defines a longitudinal rail or spine 123 that includes a pair of rail flanges 123A extending laterally outward from opposite sides of the rail 123. In the illustrated embodiment, the device coupling member 130 defines a longitudinal channel or slide track 133 that includes a pair of channel flanges 133A extending laterally inward from opposite sides of the channel 133. The rail 123 including the rail flanges 123 and the channel 133 including the channel flanges 133A are configured to laterally constrain the rail 123 within the channel 133. As such, the rail 123 may be separated from the channel 133 via longitudinal displacement of the rail 123 with respect the channel 133 while lateral separation of the rail from the channel 133 is prevented.

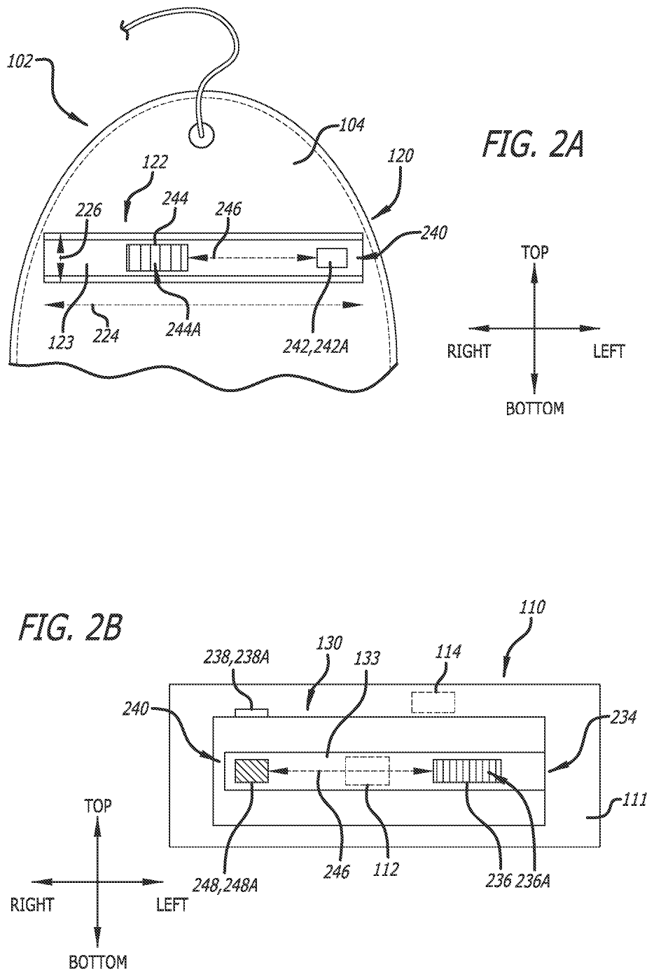

FIGS. 2A-2B illustrate further detailed views of the system 100 including further components of the coupling system 120. FIG. 2A is a back side view of a portion of the bag 102 including the bag coupling member 122 of the coupling system 120. FIG. 2B is a corresponding front view of the device 110 including the device coupling member 130 of the coupling system 120. In the illustrated embodiment, the coupling system 120, including bag coupling member 122 and the device coupling member 130, is oriented horizontally (i.e., left to right). In other embodiments, the coupling system 120 may be oriented vertically (i.e., top to bottom).

Referring to FIG. 2A the bag coupling member 122 may include a first length 224 and a first width 226. The bag coupling member 122 may extend laterally across a portion of (or an entire width of) the urine collection bag 102. The bag coupling member 122 may be constructed of any suitable material, such as a rigid plastic, aluminum, or stainless steel, for example. Similarly, the bag coupling member 122 may be attached to the bag 102 via any suitable attachment mechanism, such as adhesive bonding, solvent bonding, radio frequency welding, ultrasonic welling, or heat welding, for example. In some embodiments, the bag 102 including the bag coupling member 122 may be configured for one or more uses with a single patient or for a single use.

Referring to the FIG. 2B, in some embodiments, the device coupling member 130 (i.e., the channel 133) may be open at one end (e.g., the right end as illustrated) and closed at the other end (e.g., the left end as illustrated). In other embodiments, the device coupling member 130 may be open at both ends. In the illustrated embodiment, the device coupling member 130 defines a first opening 234.

Referring to FIGS. 2A-2B, the coupling system 120 may include a locking mechanism 240, according to some embodiments. The locking mechanism 240 is generally configured to prevent separation (i.e., decoupling) of the bag coupling member 122 from the device coupling member 130 in the absence of a deliberation action by the clinician or the system 100. More specifically, the locking mechanism 240 may prevent or restrict longitudinal displacement of the bag coupling member 122 with respect to the device coupling member 130 when the bag coupling member 122 is coupled with the device coupling member 130.

The locking mechanism 240 may include a first locking portion 242 coupled with or incorporated into the bag coupling member 122 and a second locking portion 248 coupled with or incorporated into the device coupling member 130. In some embodiments, the first locking portion 242 may include a recess 242A of the bag coupling member 122. Similarly, the second locking portion 248 may include a protrusion 248A of the device coupling member 130. As such, the recess 242A may be configured to receive the protrusion 248A, thereby preventing longitudinal displacement of the bag coupling member 122 with respect to the device coupling member 130. It can be appreciated that the first locking portion 242 may be coupled with or incorporated into the device coupling member 130 and the second locking portion 248 may be coupled with or incorporated into the bag coupling member 122.

In some embodiments, the second locking portion 248 may be deflectable between a protruding state (i.e., a non-deflected state) consistent with disposition of the protrusion 248A within the recess 242A and a non-protruding state (i.e., a deflected state) consistent with extraction of the protrusion 248A from the recess 242A. In some embodiments, the locking mechanism 240 may define a snap fit, where the second locking portion 248 self-deflects away from the non-protruding state to engage (i.e., protrude within) the recess 242A. In some embodiments, the second locking portion 248 may include or be coupled with an actuator 238 configured to transition the protrusion 248A between the protruding state and the non-protruding state. In some embodiments, the actuator 238 may include a button pressable by the clinician, so that the clinician may press the button to transition protrusion 248A from the protruding state to the non-protruding state.

In some embodiments, the actuator 238 may include an electro-mechanical actuator 238A coupled with the console 114 wherein the console 114 where logic of the console may automatically activate the actuator 238 to transition the locking mechanism 240 between the locked configuration and the unlocked configuration.

Referring again to FIGS. 2A-2B, the bag coupling member 122 may include an electronic patient identification device (ID device) 244 configured to store patient data thereon. In some embodiments, the patient data may include a patient identification, historical urine collection data, urine flow rate data, or the like. In some embodiments, the ID device 244 may include a printed circuit board (PCB) including memory and a number (e.g., 2, 3, 4 or more) of electrical contacts 244A, where the electrical contacts 244A are externally exposed.

The device coupling member 130 may include a console connector 236 electrically coupled with the console 114. In some embodiments, the console connector 236 may include connector contacts (i.e., electrical contacts) 236A disposed along the channel 133. The connector contacts 236A are configured (e.g., positioned and/or sized) to correspondingly connect to the electrical contacts 244A when the bag coupling member 122 is coupled with the device coupling member 130. As such, the console 114 may exchange data with the ID device 244. In some embodiments, the console connector 236 and the locking mechanism 240 may be separated by the first distance 246.

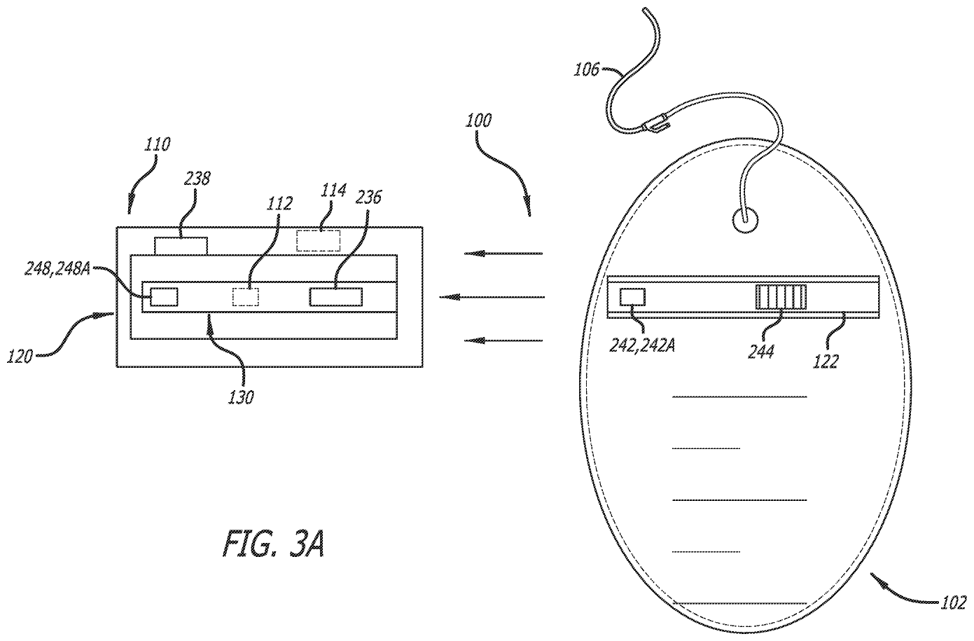

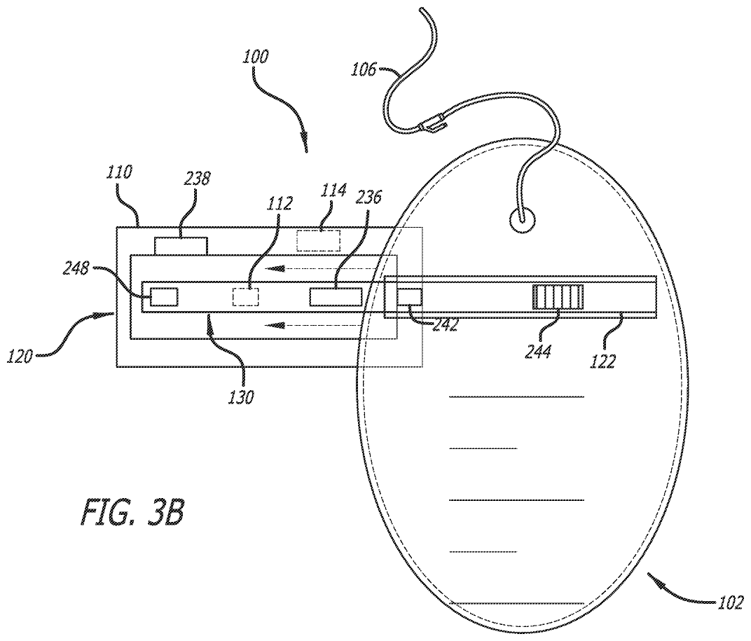

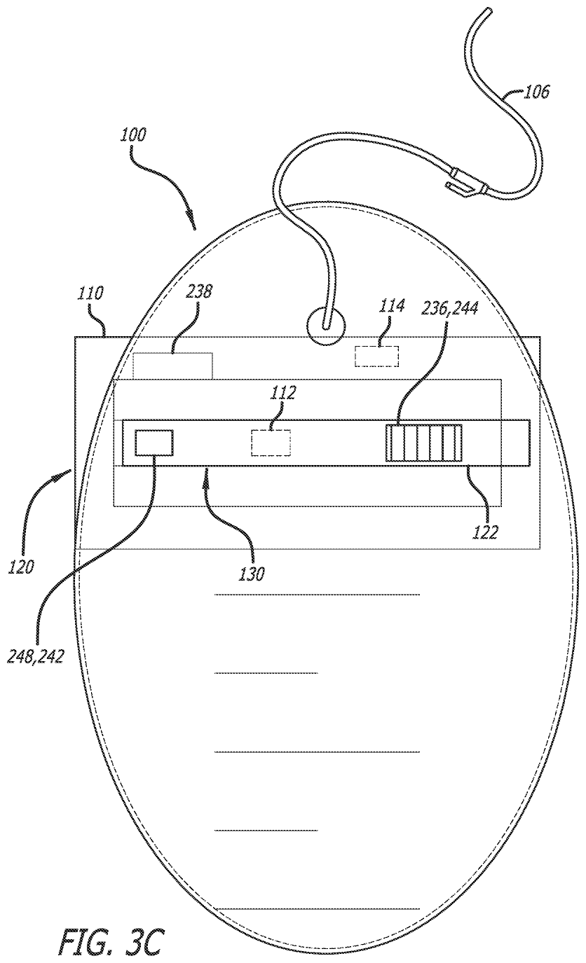

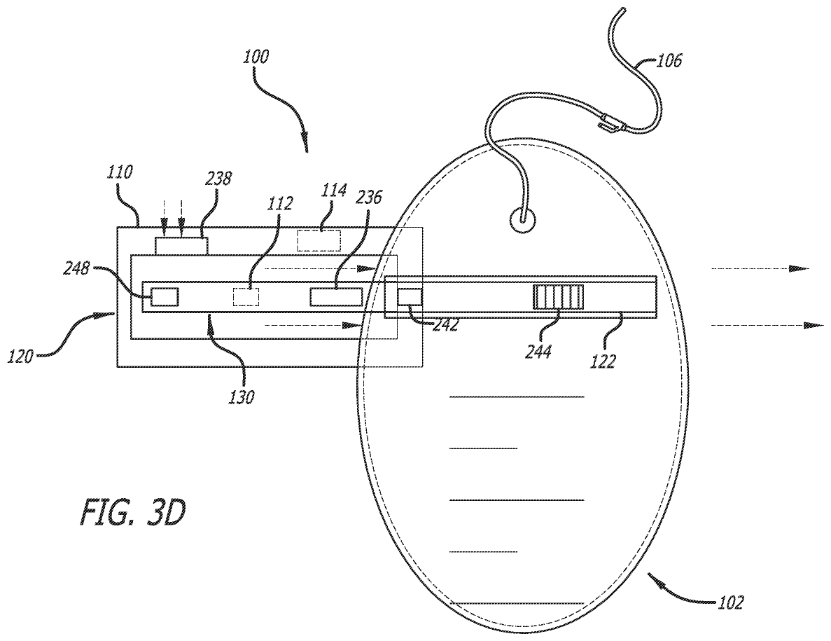

FIGS. 3A-3D illustrate front views of the system 100 in various states of coupling. FIG. 3A illustrates the bag 102 and the device 110 is a separated state. FIG. 3B illustrates the bag 102 and the device 110 is a partially coupled state. FIG. 3C illustrates the bag 102 and the device 110 is a fully coupled state, and FIG. 3D illustrates the bag 102 and the device 110 is a partially decoupled state. As shown in FIG. 3A, the bag coupling member 122 may be aligned with the device coupling member 130. With the bag coupling member 122 aligned with the device coupling member 130, the bag 102 may be displaced with respect to the device 110 to begin transitioning the coupling system 120 from the separated state to toward the coupled state. In some embodiments, the bag 102 may be coupled with a urinary catheter 106.

As shown in FIG. 3B, the bag coupling member 122 may engage the device coupling member 130 as the bag 120 is displaced with respect to the device 110. More specifically, the rail 123 may be inserted into the channel 133 (see FIG. 1).

As shown in FIG. 3C, the bag coupling member 122 may be advanced along the device coupling member 130 until the bag 102 is fully coupled with the device 110, i.e., the coupling system 120 is fully transitioned to the coupled state. As also shown in FIG. 3C, locking mechanism 240 (see FIGS. 2A-2B) is transitioned to the locked configuration. More specifically, the first locking portion 242 is positioned adjacent the second locking portion 248 such that the protrusion 248A is disposed within the recess 242A to prevent longitudinal displacement of the bag coupling member 122 along the device coupling member 130. In some embodiments, the locking mechanism 240 may be configured to provide tactile or audible confirmation feedback (e.g., a click) to the clinician when the locking mechanism 240 is fully transitioned to the locked configuration. With the bag coupling member 122 and the device coupling 130 fully coupled together, the connector contacts 236A may be fully coupled with the electrical contacts 244A so that the console 114 may exchange data with the ID device 244.

With the locking mechanism fully transitioned to the locked configuration, the connector contacts 236A may be prevented from decoupling from the electrical contacts 244A so that the data exchange between the console 114 the ID device 244 may remain uninterrupted. In some embodiments, the logic of the console 114 may monitor the data exchange with the ID device 244, and the logic may be configured to generate an alert when data exchange is interrupted. Similarly, the logic may enable activation of the load cell assembly 112, when the logic determines that the data exchange with the ID device 244 is established.

Similarly, with the locking mechanism fully transitioned to the locked configuration, operative decoupling of the load cell assembly 112 from the bag 102 may be prevented, thereby ensuring accurate weight measurement of the bag 102 by the load cell assembly 112. In some embodiments, the logic of the console 114 may monitor the weight measurement by the load cell assembly 112, and the logic may be configured to generate an alert when the load measurement is interrupted.

As shown in FIG. 3D, transitioning the locking mechanism 240 from the locked configuration to the unlocked configuration, may include activating the actuator 238 (e.g., pressing the button) to extract the protrusion 248A from the recess 242A (see FIGS. 2A-2B). With the locking mechanism transitioned to the unlocked configuration, the bag coupling member 122 (including the bag 102) may be displaced with respect to the device coupling member 130 (including the device 110). Initial longitudinal displacement of the bag coupling member 122 with respect to the device coupling member 130 toward the decoupling state may cause decoupling of the connector contacts 236A from the electrical contacts 244A, thereby decoupling the console 114 from the ID device 244. Continued longitudinal displacement of bag coupling member 122 with respect to the device coupling member 130 may extract the rail 123 from the channel 133 (see FIG. 1), thereby fully separating the bag 102 from the device 110.

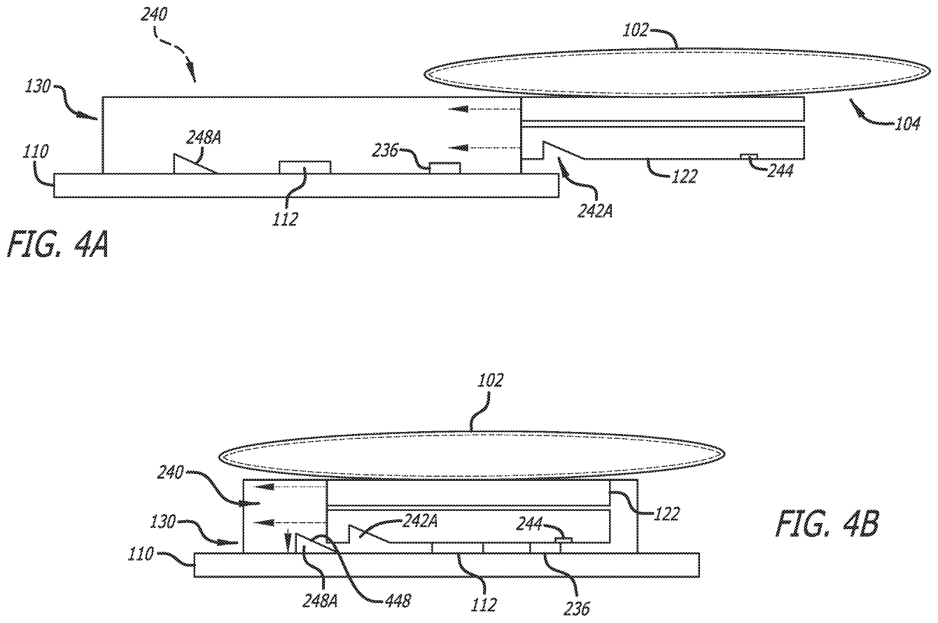

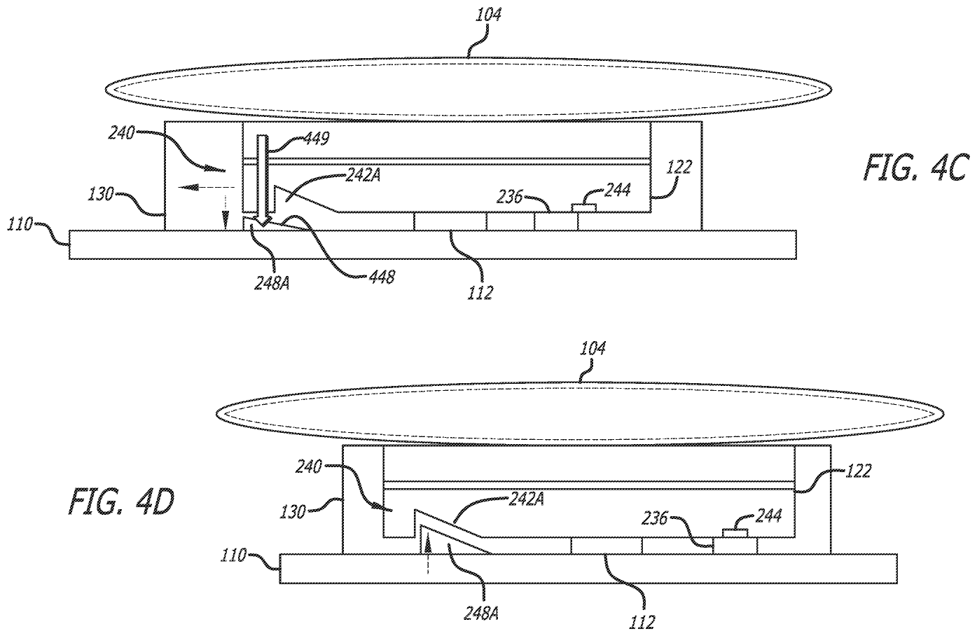

FIGS. 4A-4D illustrate a cross-sectional top views of the locking mechanism 240 transitioning between the unlocked configuration and the locked configuration. The bag coupling member 122 is coupled to the back side 104 of the bag 102 and the device coupling member 130 is coupled to the device 110 having the load cell assembly 112. The bag coupling member 122 includes the ID device 244 and the recess 242A, as illustrated in FIG. 4A. The bag coupling member 122 may longitudinally slide along the device coupling member 130 until the bag coupling member 122 engages an inclined surface 448 of the protrusion 248A, as illustrated in FIG. 4B. Continued advancement of the bag coupling member 122 along the device coupling member 130 causes the bag coupling member 122 slide along the inclined surface 448, thereby applying a deflection force 449 to the protrusion 248A. The deflection force 449 defects the protrusion 248A toward to the non-protruding state, as illustrated in FIG. 4C. The bag coupling member 122 may continue to slide along the device coupling member 130 until the protrusion 248A is disposed adjacent the recess 242A, whereupon the protrusion 248A self-deflects into the recess 242A to fully transition the locking mechanism 240 into the locked configuration. With the locking mechanism 240 transitioned to the locked configuration, the electrical contacts 244A are couple with the connector contacts 236A coupled so that the ID device 244 is coupled with the console 114.

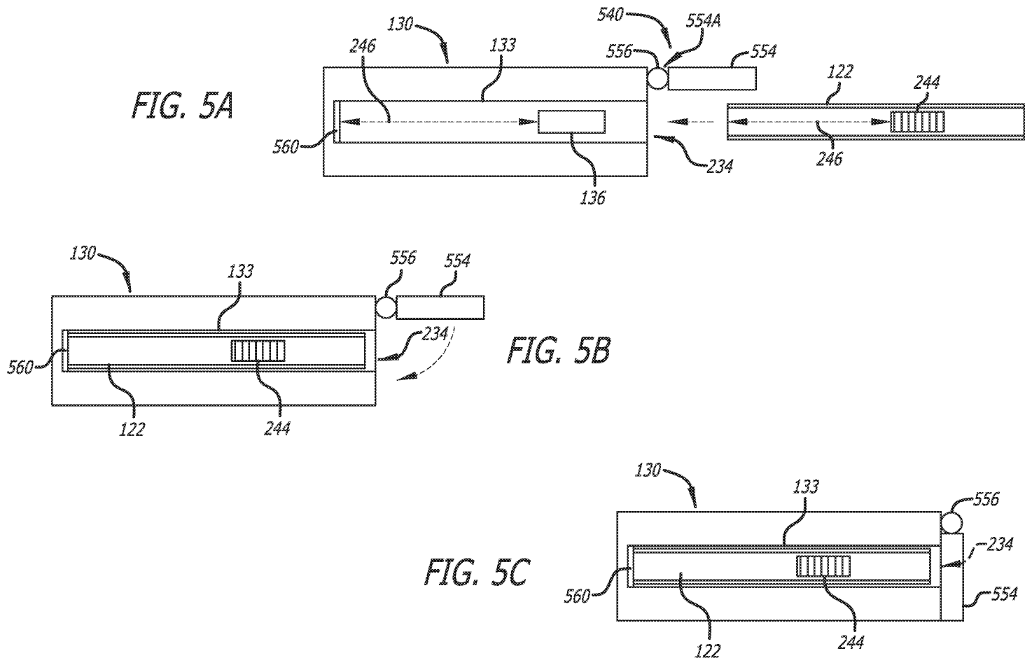

FIGS. 5A-5C illustrate another embodiment of a locking mechanism 540 that may in certain resemble the components and functionality of the of the locking mechanism 240. FIG. 5A illustrates the locking mechanism 540 in an unlocked configuration with the bag coupling member 122 separated from the device coupling member 130. The locking mechanism 540 includes a latch member (e.g., a door or gate) 554 coupled to the device coupling member 130 by a hinge 556.

FIG. 5B illustrates the locking mechanism 540 in the unlocked configuration with the bag coupling member 122 fully coupled with the device coupling member 130. In some embodiments, the device coupling member 130 may include the bumper 560 configured to prevent additional movement of the bag coupling member 122 in the direction opposite the first opening 234.

FIG. 5C illustrates the locking mechanism 540 in a locked configuration with the bag coupling member 122 fully coupled with the device coupling member 130. The latch 554 is configured to transition between (i) an unlocked position consistent with the locking mechanisms 540 disposed in the unlocked configuration and (ii) a locked position consistent with the locking mechanisms 540 disposed in the locked configuration. As shown, the latch member 554 is configured to inhibit longitudinal displacement of the bag coupling member 122 away from a fully coupled state with the device coupling member 130 when the latch member 554 is disposed in the locked position. In some embodiments, the latch member 554 may extend away from the first opening 234 (e.g., be oriented parallel to the channel 133) in the unlocked position. Similarly, the latch member 554 may extend across the first opening 234 (e.g., be oriented perpendicular to the channel 133) in the locked position.

In some embodiments, the latch 554 may be manually transitioned from the unlocked position to the locked position. In other embodiments, the latch 554 may include an electro-mechanical actuator 554A. In some embodiments, electro-mechanical actuator 554A may be a rotational actuator coupled with or incorporated into the hinge 556. The electro-mechanical actuator 554A may be coupled with the console 114 so that logic of the console may automatically activate the actuator 554A to transition the latch member 554 between the locked position and the unlocked position.

In some embodiments, the logic of the console 114 may automatically transition the latch member 554 from unlocked position to the locked position upon determining that the ID device 244 is coupled with the console 114. In some embodiments, the clinician may prompt the logic to transition the latch 554 from the locked position to the unlocked position. In an embodiment, the logic may transition the latch 554 from the locked position to the unlocked position when logic determines that the bag 102 has collected a certain volume of urine therein. In some embodiments, the latch 154 may include a securing mechanism (not shown) to secure the latch member 554 to the device coupling member 130 in the locked position.

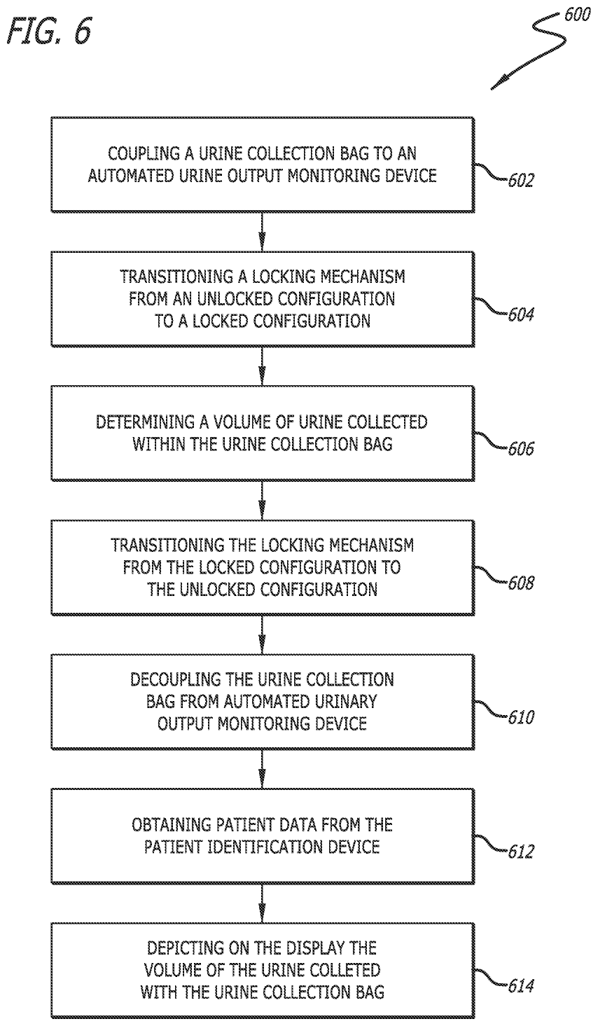

FIG. 6 illustrates a flow chart of an exemplary method 600 of monitoring urinary output that, in accordance with some embodiments, may include all or any subset of the following steps, actions, or processes. In some embodiments, the method 600 may include coupling the urine collection bag 102 to the automated urinary output monitoring device 110 using the coupling system 120 (block 602). In some embodiments, coupling the urine collection bag 102 to the automated urinary output monitoring device 110 using the coupling system 120 includes the device coupling member 130 coupled to the automated urinary output monitoring device 110 slidably receiving the bag coupling member 122 coupled to the urine collection bag 102. In some embodiments, detachably coupling the urine collection bag 102 to the automated urinary output monitoring device 110 includes the urine collection bag 102 being in fluid communication with the urinary catheter 106. In some embodiments, coupling the urine collection bag to the automated urinary output monitoring device 110 includes slidably advancing the rail 123 within the channel 133.

The method 600 may further include transitioning the locking mechanism 240 from the unlocked configuration to the locked configuration (block 604). In some embodiments, transitioning the locking mechanism 240 from the unlocked configuration to the locked configuration includes the protrusion 248A being received by the recess 242A to prevent additional movement of the bag coupling member 122 within the device coupling member 130. In some embodiments, the protrusion 248A protrudes away from the device coupling member 130 and the recess 242 is located on the bag coupling member 122. In some embodiments, transitioning the locking mechanism 240 from the unlocked configuration to the locked configuration includes the protrusion 248A self-deflecting into the recess 242. In some embodiments, in the locked configuration, the ID device 244 is coupled with the console 114.

The method 600 may further include determining the volume of urine collected within the bag 102 (block 606). In some embodiments, determining the volume of urine includes detecting (measuring) load defined by a weight of the urine collection bag 102. In some embodiments, detecting the load of the urine collection bag 102 includes obtaining the load values as the urine collection bag 102 receives the urine therein. In some embodiments, detecting the load includes transmitting load data to the console 114. In some embodiments, the load corresponds to a volume of urine collected within the urine collection bag 102. In some embodiments, detecting the load includes obtaining multiple load values according to a user defined time interval (e.g., every 15 minutes, every hour, or the like).

The method 600 may further include transitioning the locking mechanism 240 from the locked configuration to the unlocked configuration (block 608). In some embodiments, transitioning the locking mechanism 240 from the locked configuration to the unlocked configuration includes using the actuator 138 to transition the locking mechanism 240 from the locked configuration to the unlocked configuration. In some embodiments, transitioning the locking mechanism 240 from the locked configuration to the unlocked configuration includes removing the protrusion 248A from the recess 242A to allow the bag coupling member 122 to slide out of the device coupling member 130. The method 600 may further include decoupling the bag 102 from the device 110 (block 610).

The method 600 may further include obtaining patient data from the patient identification device 244 (block 612). In some embodiments, obtaining patient data establishing an electrical connection between the having a printed circuit board that includes a number of electrical contacts 244A disposed along the rail 123 and the console 114 via the console connector 236 having a number of connector contacts 236A disposed along the channel 133. Once connected, the logic of the console 114 may obtain patient data from the patient identification device 144.

The method 600 may further include depict the volume of urine collected within the bag 102 on the display 116 (block 614). In such embodiments, the logic of the console may depict the volume of urine on the display 116.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. An automated urinary output monitoring system, comprising:
a urine collection bag configured to collect urine excreted by a patient via a urinary catheter;
an automated urinary output monitoring device, comprising:
a load cell assembly operatively coupled with the urine collection bag, the load cell assembly configured to measure a load defined by urine collected within the urine collection bag;
a console coupled with the load cell assembly, the console including a processor and a non-transitory computer readable medium having logic stored thereon that, when executed by the processor, performs operations that include determining a volume of the urine collected with the urine collection bag based on the load; and
a coupling system configured to detachably couple the urine collection bag to the automated urinary output monitoring device, the coupling system including:
a rail attached to one of the urine collection bag or the automated urinary output monitoring device; and
a channel attached to the other one of the urine collection bag or the automated urinary output monitoring device, the channel configured to (i) longitudinally and slidably receive the rail and (ii) prevent lateral separation of the rail from the channel.

2. The system according to claim 1, wherein the channel is attached to a front side of the automated urinary output monitoring device and the rail is attached to a back side the urine collection bag.

3. The system according to claim 2, wherein the rail extends laterally across a portion of a width of the urine collection bag.

4. The system according to claim 2, wherein the channel extends laterally across the front side of the automated urinary output monitoring device.

5. The system according to claim 1, wherein the channel includes an open end and a closed end.

6. The system according to claim 5, wherein the channel includes a bumper at the closed end, the bumper configured to abut the rail when the rail is received within the channel.

7. The system according to claim 1, wherein the coupling system includes a locking mechanism configured to transition between an unlocked configuration and a locked configuration such that:
longitudinal displacement of the rail with respect to the channel is restricted when the locking mechanism is transitioned to the locked configuration, and
longitudinal displacement of the rail with respect to the channel is allowed when the locking mechanism is transitioned to the unlocked configuration.

8. The system according to claim 7, wherein the locking mechanism includes a deflectable protrusion coupled with the channel and a recess disposed within the rail, the recess configured to receive the protrusion when the locking mechanism is transitioned to the locked configuration, thereby restricting longitudinal displacement of the rail with respect to the channel.

9. The system according to claim 8, wherein the locking mechanism includes an electro-mechanical actuator operatively coupled between the deflectable protrusion and the console, the operations further including activating the electro-mechanical actuator to:
transition the locking mechanism from the unlocked configuration to the locked configuration, and
transition the locking mechanism from the locked configuration to the unlocked configuration.

10. The system according to claim 7, wherein:
the locking mechanism includes a latch member rotatably coupled to the channel at the open end,
the latch member is rotated so as to extend across the open end when the locking mechanism is transitioned to the locked configuration,
the latch member is rotated so as to extend away from the open end when the locking mechanism is transitioned to the unlocked configuration.

11. The system according to claim 10, wherein locking mechanism includes an electro-mechanical actuator operatively coupled between the latch member and the console, the operations further including activating the electro-mechanical actuator to:
transition the locking mechanism from the unlocked configuration to the locked configuration and
transition the locking mechanism from the locked configuration to the unlocked configuration.

12. The system according to claim 1, wherein the rail includes a patient identification device coupled thereto, the patient identification device having patient data stored in memory thereon.

13. The system according to claim 12, wherein:
the patient identification device includes a printed circuit board including a number of electrical contacts disposed along the rail,
the channel includes a console connector coupled with the console, the console connector including a number of connector contacts disposed along the channel, and
the electrical contacts and the connector contacts are configured to correspondingly couple with each other when the urine collection bag is coupled with the automated urinary output monitoring device to enable data exchange between the console and patient identification device.

14. The system according to claim 1, further comprising a display coupled with the console, the operations further include depicting on the display the volume of the urine collected with the urine collection bag.

15. A method of monitoring urinary output, comprising:
coupling a urine collection bag to an automated urinary output monitoring device;
transitioning a locking mechanism from an unlocked configuration to a locked configuration, the locking mechanism configured to prevent decoupling of the urine collection bag from the automated urinary output monitoring device in the locked configuration;
determining a volume of urine collected within the urine collection bag;
transitioning the locking mechanism from the locked configuration to the unlocked configuration; and
decoupling the urine collection bag from the automated urinary output monitoring device.

16. The method according to claim 15, wherein coupling the urine collection bag to the automated urinary output monitoring device includes slidably advancing a rail attached to the urine collection bag longitudinally within a channel attached to the automated urinary output monitoring device, the channel configured to prevent lateral separation of the rail from the channel.

17. The method according to claim 16, wherein the channel is attached to a front side of the automated urinary output monitoring device and the rail is attached to a back side the urine collection bag.

18. The method according to claim 16, wherein the locking mechanism is configured to:
restrict longitudinal displacement of the rail within the channel when the locking mechanism is transitioned to the locked configuration, and
allow longitudinal displacement of the rail within channel when the locking mechanism is transitioned to the unlocked configuration.

19. The method according to claim 18, wherein the locking mechanism includes a deflectable protrusion coupled with the channel and a recess disposed within the rail, the recess configured to receive the protrusion when the locking mechanism is transitioned to the locked configuration, thereby restricting longitudinal displacement of the rail within the channel.

20. The method according to claim 16, wherein:
the locking mechanism includes a latch member rotatably coupled to the channel at an open end of the channel opposite a closed end of the channel,
the latch member is rotated so as to obstruct the open end when the locking mechanism is transitioned to the locked configuration, thereby securing the rail within the channel, and
the latch member is rotated so as to extend away from the open end when the locking mechanism is transitioned to the unlocked configuration, thereby allowing extraction of the rail from the channel via the open end.

21. The method according to claim 15, wherein:
the urine collection bag includes a patient identification device having a printed circuit board that includes a number of electrical contacts disposed along the rail,
the automated urinary output monitoring device includes a console coupled with a console connector having a number of connector contacts disposed along the channel,
the electrical contacts and the connector contacts are configured to correspondingly couple with each other when the urine collection bag is coupled with the automated urinary output monitoring device to enable data exchange between the console and patient identification device, and
the method further includes obtaining patient data from the patient identification device.

22. The method according to claim 15, wherein the automated urinary output monitoring device includes a display coupled with the console, the method further comprising depicting on the display the volume of the urine collected with the urine collection bag.
